(19) United States
(12) Patent Application Publication
(10) Pub. No.: US 2011/0275959 A1
(43) Pub. Date: Nov. 10, 2011

(54) PORTABLE SYSTEM FOR MONITORING THE POSITION OF A PATIENT'S HEAD DURING VIDEONYSTAGMOGRAPHY TESTS (VNG) OR ELECTRONYSTAGMOGRAPHY (ENG)

(76) Inventors: Henry Eloy Sand Casali, Buenos Aires (AR); Miguel Angel Lacour Argerich, Capital (AR); Marcos Maria Ledesma Williams, Pilar (AR); Guillermo Horacio Diez, Mar del Plata (AR)

(21) Appl. No.: 13/085,153

(22) Filed: Apr. 12, 2011

Related U.S. Application Data
(63) Continuation-in-part of application No. 11/895,831, filed on Aug. 28, 2007, now abandoned.
(60) Provisional application No. 60/841,096, filed on Aug. 30, 2006.

(51) Int. Cl. A61B 5/103

(52) U.S. Cl. 600/595

(57) ABSTRACT

A system and a method is presented that includes monitoring in real time the position and movement of the head with regard to a visual stimulator during videonystagmography (VNG) or electronystagmography (ENG) studies in a patient. Ultrasound sensors and emitters are used combined with gyroscopes as gravity detectors, or other devices. The signals of the sensors are processed by software that carries out positioning monitoring that facilitates the operation of the operator and makes a quality control of the diagnostic maneuvers, thus providing a test reproducible method for patients. Reproducibility is ensured by means of acceptance limits provided by the software. The invention provides a way of ensuring the reproducibility according to ANSI standard in the tests by guiding the operators throughout the process so that they may conduct the tests in the same way for all patients. This inventive portable system can be use in any physician's office.

### CROSS REFERENCE TO RELATED APPLICATION

The present application is a continuation-in-part of co-pending U.S. patent application Ser. No. 11/895,831 filed on Aug. 28, 2007, and claims the benefit of priority of U.S. Provisional Application No. 60/841,096 dated Aug. 30, 2006, the entire contents and disclosures of which are incorporated herein by reference.

### FIELD

This invention is under the scope of equipment employed in medicine, specially in relation to videonystagmography tests.

### BACKGROUND

Videonystagmograph (VNG) and electronystagmograph (ENG) are equipment known for several years.

Their specific function is the diagnosis of equilibration, balance and vertigo problems of a person by testing the interrelation among the middle ear, the brain and the eye movements.

The diagnosis is based on the stimulation of the balance system, by means of a special positioning of the head, heating/cooling of the middle ear, head movements or visual stimulation of the patient and his response through eye movements, specially nystagmus.

The different equipment manufacturers have developed techniques and software to stimulate and record responses both in analogical and digital form.

Although some progress has been achieved in providing measurements of distances and/or speeds, there is still no system which can be consistent in its operation, therefore, the tests made by different technicians are not comparable. On the other hand, tests are not quality controlled.

The present results of the tests depend totally on the training, education and care of the technician or professional that carries out the test.

The dimensions of the equipment available in the market make it impossible to move them, limiting their use to the physician's offices, in view of the fact that they require permanent installations with mountings and fittings which prevent them from being moved.

It may be seen that document U.S. 2007/0161875 (A1) by Epley et al. (hereinafter “Epley”) relates to a structure and methodology involving a mountable and head-wearable frame structure, which during use is positionally stabilized relative to a human subject's head. The structure carries a selection of positionally anchored data sensors and stimuli deliverers, which are relevant to the diagnosis and treatment of vestibular disorders. Special configurations are provided for two types of stimulators, as follows: sound application and air-pressure modification, and introduction of fluids to the ear. Stabilization enables tight and accurate correlation of data which is analyzable by a connected, properly algorithmed computer, that can also be used for a feedback control. The invention enables a practical and significant differentiation between physiological and pathological nystagmus.

It may be seen from this document that Epley's work is directed to the search of new diagnoses and treatments, particularly emphasizing the physician's participation. It is significantly important that the practitioner is qualified and capable of carrying out the tests in a reproducible way so that the diagnoses will be useful. In view of the fact that it is the practitioner who determines the parameters and controls the test, the application of rules is subjected to the professional knowledge and skill Therefore, there is no alarm system in the equipment employed.

In the equipment described in Epley, two accelerometers and one inclinometer are used, as a consequence of which only the posture tests and Epley's maneuvers may be comprised therein.

Therefore, it is desirable to have a system that enables the operator's tests to be independent, so that any moderately trained technician may carry them out. It is necessary then to have a system of alarm-associated detectors that enables the operator and the patient to carry out the tests with a diagnostic value by following the rules in force and achieving a maximum standardization of vertigo tests.

It is also desirable to be able to carry out at the same time with the same system the posture tests together with the optokinetic tests, for which a positioning and monitoring system of the movements of a patient's head is necessary.

Likewise, it is also desirable to have a system that may be used as an “up-grade” of the existing videonystagmographs (VNG) and the electronystagmographs (ENG), in view of the fact that they are expensive and difficult to renew in short-cycle terms.

### SUMMARY OF THE INVENTION

In order to overcome the disadvantages of the state of the art, the inventors present a portable system of detection in real time of the three-dimensional position and the speed of movement of a person's head subject to a test of videonystagmography, along with software that uses this positioning system to guide the technicians and practitioners that use the videonystagmography equipment to perform the different maneuvers of positioning and/or rotation of the patient's head, thus ensuring that the tests be made in a correct, reproducible and consistent way in accordance with the “Procedures for Testing Basic Vestibular Function” of the American National Standards Institute, Inc. and The Acoustical Society of America (ASA), ANS/ASA S3.45-2009 (Revision of ANSI S3.45-1999).

The portable equipment is carried in a briefcase and connected to a computer, which may also be portable such as a “laptop” or “notebook” or a personal computer (PC) via an external serial communication.

Accordingly, this is a portable system that may be easily carried and which may be used in any physician's office. The system is easy to move in view of the fact that it may be placed into a briefcase.

### BRIEF DESCRIPTION OF THE FIGURES

FIG. 1 shows a global block diagram of a preferred embodiment of the system of the present invention.

FIG. 2 shows a preferred embodiment of the system of the present invention with details of the basic components.

FIG. 3 shows a preferred embodiment of the goggles VNG in the system of FIG. 2.

FIG. 4 shows the different types of monitoring of the position and movements of a patient's head, made by the system according to this invention.

FIG. 5 shows a block diagram that displays the interaction between the operator and the patient during the VNG or ENG tests through the system of this invention.

FIG. 6 shows a block diagram that gives details of the components of the system according to the present invention.

FIG. 7 shows a block diagram which describes in detail the registration process and the data transmission to the computer.

FIG. 8 shows a block diagram which describes the signaling process that may be carried out by the system of the present invention.

FIG. 9 shows a block diagram of the oculomotor or optokinetic tests that may be carried out with the system according to the present invention.

FIG. 10 shows a block diagram of the postural tests that can be carried out with the system according to the present invention.

FIG. 11 shows a block diagram of the caloric stimulation tests that can be carried out with the system according to the present invention.

FIG. 12 shows a block diagram of the rotatory stimulation tests (Active Head Rotatory (AHR)) that may be carried out with the system according to the present invention.

FIG. 13 shows the screen where the Software verifying the position of the patient opposite the visual stimulator in real time warns the operator to correct the operation as necessary.

FIG. 14 shows the screen where the Software verifying the angular position of the patient's head in real time warns the operator to correct the operation as necessary.

FIG. 15 shows the screen where the Software verifying in real time the position of the head in relation to the visual stimulator and the angular motion of the patient's head with alarm (in red) of movements or positioning outside the reproducibility range, and which also monitors the eye movement in real time and makes a chart of the eye movement in a horizontal and vertical channel, warns the operator to correct the operation as necessary.

### DETAILED DESCRIPTION OF THE INVENTION

Dizziness and problems of balance, including vertigo, are one of the most common causes of complaints received by the medical professionals at present.

There exist many causes and abnormalities that produce these symptoms. These causes can be affecting the central nervous system or the peripheral one, but most of the problems derive from the inner ear organs involved in the detection of the position and movement of the head, and there can also exist problems related to the nerves that communicate the inner ear with the brain, vascular problems, blood flow in the spine, traumatisms, etc.

The inner ear contains semicircular channels, which orthogonal space orientation allows the record of the position, speed and acceleration of the head. The information obtained by this organ generates automatic reflections and is in turn processed together with other somatosensorial and visual signals that the central nervous system gathers to obtain the balance, stability, or sensation of movement, acceleration, etcetera.

The information is fed back to the eye muscles for its positioning through the vestibular ocular reflex (VOR) which is the one that permanently coordinates the relative position detected by the semicircular channels of the internal or inner ear with the position of the eyes.

The diagnosis of problems in the system of detection of both position and balance is based on the observation of the eye movements that respond to the VOR.

In case of abnormal detection of position, speed or acceleration by the semicircular channels or by the nervous system, the eyes will respond abnormally trying to adapt to an abnormal position, this will produce a movement of adaptation of the eye called nystagmus.

Nystagmuses are classified as normal and abnormal, the normal ones respond to real stimuli and true detected positions, and the abnormal ones respond to positions detected erroneously by the vestibular or nervous system and do not correspond to real positions, speeds or accelerations.

The detection and analysis of the nystagmus together with controlled stimuli provide valuable information for the diagnosis of problems in the inner ear and/or central nervous system and/or the communication between both of them.

The exploration of the vestibular system requires the VOR, the function of the cerebellum and the stability evaluation.

This involves a great number of tests to explore the different parts of the vestibular system and the neurological structures involved in the maintenance of the balance.

The videonystagmography is part of the set of tests that allow the exploration of the vestibular system. This consists basically of different sub-groups of tests:
Oculomotor or Optokinetic (Saccades, Smooth Pursuit, OKN, Gaze and Spontaneous Nystagmus) tests.
Postural and Positional tests (Positional and positioning, DIX-Hallpike Maneuvers).
Caloric Stimulation test.
Active Head Movements (Head Trust) test.
Head rotation (Active Head Rotatory (AHR)/Rotatory) test.
Spontaneous tests

All the tests are evaluated by recording the eye movements, which in this particular case is the nystagmus.

Each test is carried out by means of different stimulation techniques that allow the analysis of the responses of the different components of the vestibular system. These techniques or protocols differ greatly from each other, and show an important amount of variables to be controlled; these can be error sources in the tests, their response being intrinsically dependant on the state of the variables to be controlled.

Due to the complexity and diversity of the tests to be undertaken, it is necessary to control a great number of variables, which implies the need for highly qualified personnel.

The aim of the portable system according to the present invention is to automatically monitor these variables to:
Standardize the conduction of the tests and, therefore, increase the reliability of the results.
Standardize protocols and generalize the analysis, producing reliable common patterns that facilitate the creation of compatible data bases to obtain new conclusions of diagnosis.
Significantly reduce the curve of training of the personnel in charge of conducting the tests by means of an expert system capable of producing by means of audio and visual presentation, the orders necessary to make the tests.
Ability to measure these parameters with a portable system, allowing the conduction of massive tests on patients.

The standardization of tests and protocols is supported, for example, by the “Procedures for Testing Basic Vestibular Function” of the American National Standards Institute, Inc. and The Acoustical Society of America (ASA), ANS/ASA S3.45-2009 (Revision of ANSI 53.45-1999), which describe how the techniques must be performed by the respective practitioners in such a way that the tests be always carried out in the same way and be comparable to each other so that the obtained results be meaningful and useful for diagnosis.

The tests that can be conducted with the system of the present invention are the following:

### Oculomotor Or Optokinetic Tests

They are made by means of visual stimuli produced in a bar or monitor called visual stimulator.

The patient must be seated, straight and with his head steady. Stimuli must be generated to known amplitudes or eccentricity degrees. This is achieved by placing the patient at a distance d from the screen:

d=eccentricity/tan (α_{desired})

Since the technique is standardized, you must check that the patient's head does not move and the distance is adequate to produce the stimulation required for the different tests.

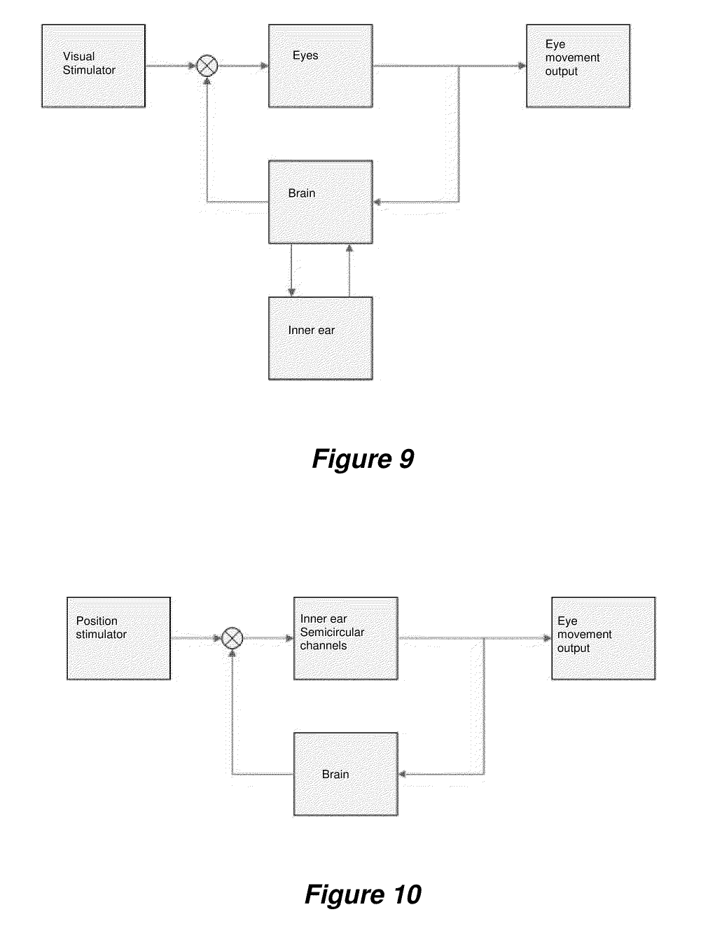

The block diagram in FIG. 9 schematically shows the operation of the oculomotor or optokinetic tests that are conducted with the system according to the present invention.

The visual stimulator produces different types of stimuli, a point that comes and goes horizontally or vertically with an even speed or that always moves repeatedly towards the same direction, or a programmed stimulus.

The visual stimulus is caught by the eyes which send the signal to the brain which acts on the eye muscles to obtain its positioning thus producing the follow-up of the stimulus.

If the eyes follow the stimulus correctly then the feedback system works properly. In the case that the eyes do not follow the stimulus correctly or the patient reports dizziness, this is due to a fault in the feedback and/or interrelation between the eyes, the brain and its relation to the inner ear which will be expressed in abnormal nystagmuses, in connection with dizziness or other symptoms detected by the system.

Therefore, the distance, height and laterality of the patient's head are followed by ultrasound while the movements and inclination of the head are monitored by means of a gyroscope, which enables the control of the test by an alarm system.

### Postural And Positioning Tests Or DIX-Hallpike Maneuvers

The block diagram in FIG. 10 schematically shows the operation of the Postural and Positioning tests that are conducted with the system according to the present invention.

In order to determine if the nystagmus origin is postural (positional) or positional (positioning), it is required to explore the patient in certain positions.

The nystagmus that appears as a result of a movement of the head or the head and the body is said to be a positional nystagmus (positioning); however, if it occurs in a certain position of the steady head, it is of a postural origin (positional).

For example, when the patient is sitting, the head is turned approximately 90° to the right and is abruptly taken to the supine position with the turned head; after a few seconds it is returned to the starting point. The maneuver is repeated but with the head turned about 90° towards the left.

In the case of postural tests, the patient takes certain positions of the head with the aid of the operator. These positions are detected by the semicircular channels of the inner ear and, upon being processed by the brain, they make the eyes adapt to the detected position.

If any problem occurs in this process, the output of the eye movement will not have any correlation with the stimulus position and the eyes will look for unreal positions, that generate abnormal nystagmus.

In this case, the speed, movements and inclination of the patient's head are followed by a gyroscope with which the control of tests is achieved and which is associated with an alarm system that warns when the test exceeds the provisions of the rules.

### Caloric Stimulation Tests

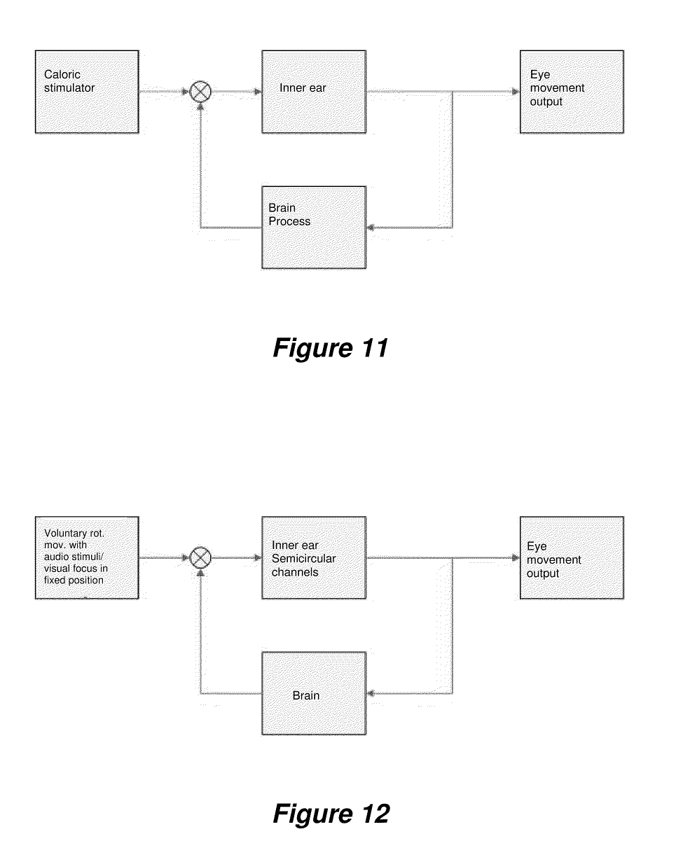

The block diagram in FIG. 11 schematically shows the operation of the caloric stimulation tests that are conducted with the system according to the present invention.

The caloric tests are based on the stimulation of the external channel of the ear by means of thermal changes with hot or cold air or water.

Heat or cold is transmitted through the tympanic membrane and the middle ear, reaching the inner ear, particularly the semicircular channels that, when varying their temperature, vary their nervous response, mainly due to the change that the temperature produces in the internal liquid of the channels. When the viscosity of this liquid varies based on the temperature, its mobility varies too and, therefore, the stimulus that it produces inside the channels.

When the patient gets dizzy because of the stimulus, the brain tries to compensate the eye position according to the information received from the semicircular channels. When these signals are altered by the caloric stimulation, nystagmus takes place, which normality or abnormality will depend on the degree of the stimulus received.

The optimal position for the stimulation of the semicircular channels involves the patient lying down with his head at about 30° over the horizontal position. In this way the semicircular channel takes a vertical position. The position of the patient's head at about 30° over the horizontal position is determined by a detector of gravity vector, for example a gyroscope.

Therefore, temperature variations produce nystagmus, which are finally determined.

### Head Active Movement Tests (Head Trust)

These tests are carried out by hand-forcing an active movement of the patient's head in order to determine either the limit of the individual or the existence of injuries.

The control is made by means of a gyroscope thus providing an alarm-system to standardize tests, that is to say to ensure that the tests be carried out in accordance with the established protocol.

The dizziness is recorded in three-dimensions by the eye movement.

### Rotatory Tests of the Head (Active Head Rotation (AHR)/Rotatory)

The block diagram in FIG. 12 schematically shows the operation of the rotatory stimulation tests that are conducted with the system according to the present invention.

It is based on the use of absolutely physiological rotatory stimuli, represented by voluntary turns of the head keeping the look fixed at a special target point.

The individual must make a sequence of movements towards the left and right sides with an increasing frequency as indicated by the stimulation system.

When making the voluntary rotatory movements in response to auditory stimuli, the inner ear detects said movements and informs the brain which sends compensation signals to the eyes.

The system three-dimensionally records the voluntary movement of the patient's head upon an auditory stimulus. It is controlled, so that no inclinations occur beyond the established standard, by means of a gyroscope as a detector, which is associated with the alarm-system.

The eye movement analysis determines if the correction measured by the ear and processed by the brain is normal, or responds to pathological parameters.

### Spontaneous Tests

In these tests the position of the patient should be at about 90° from the floor, this position being determined by a detector of gravity vector, for example a gyroscope.

There are Spontaneous Tests with patient's eyes fixation and without patient's eyes fixation, the latter comprising the use of a mask on the front portion of the goggles to cover the patient's eyes to achieve the test, then nystagmus are generated and measured.

To achieve Spontaneous Tests with patient's eyes fixation, a visible point as reference is placed on the visual stimulator screen, while the patient's head position and/or inclination is determined by a detector of gravity vector, for example a gyroscope, and the angular position and patient's head distance from the visual stimulator is determined by ultrasound generated by an ultrasound emitter mounted on the goggles, which generates an ultrasonic signal detected by ultrasound receivers of the visual stimulator.

It may be clearly seen from the above-given description that with the system of the present invention, it is not necessary to have a trained practitioner or complex equipment to carry out the above-described tests keeping within the parameters established as acceptable to deem the test as of a diagnostic value.

The system of the present invention comprises control and alarm mechanisms so that by means of ultra-sound detectors and at least a gyroscope, it is possible to determine the precise moment in which the rule is not being complied with; that is to say when the standard established for each test is exceeded. The system of the present invention does not need a trained practitioner to validly carry out these tests. It is only necessary that a technician follows the basic instructions to obtain valuable results from a diagnostic point of view.

Therefore, with the system of this invention, it is not necessary to have an expert since the system itself controls the tests by means of alarms that sound when the ultra-sound detector and/or the gyroscope detect differences with regard to the standards fixed for each test.

### Description of the System

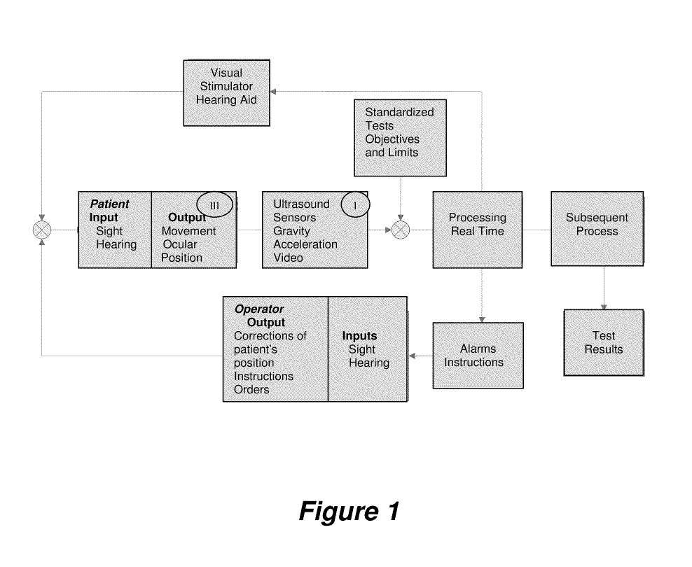

FIG. 1 shows a general block diagram of the system according to the present invention with the interrelation between the patient and the operator through the system, where the operator is a moderately trained person or a person without a deep knowledge of the technique.

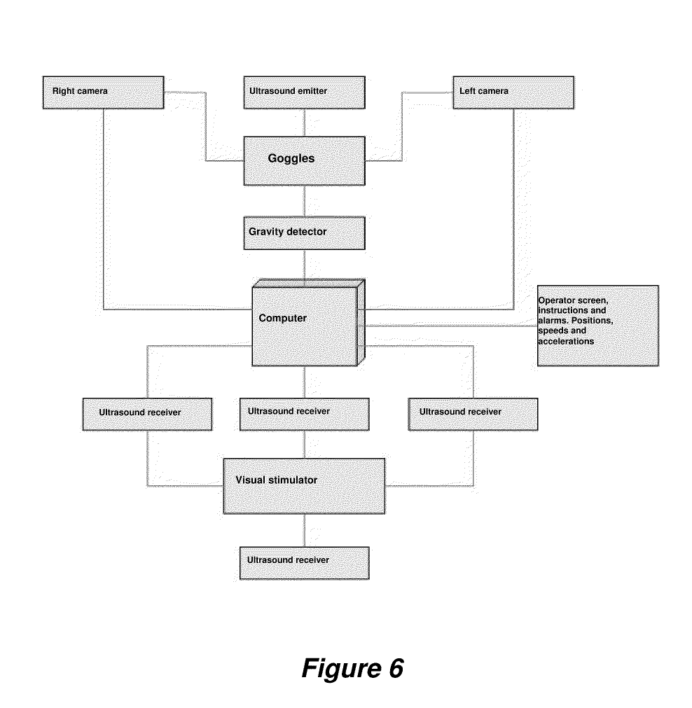

In FIG. 6, a block diagram may be seen that gives details of the components of the system according to the present invention and its interrelation.

Specifically, the portable system for the monitoring of the patient's head positioning during videonystagmography (VNG) and/or electronystagmography (ENG) studies according to this invention allows determining the position of the patient's head in relation to a visual stimulator placed at the front of his head, the patient's head inclination in relation to his vertical axis (normal) and the patient's head rotation in relation to his vertical axis (normal), where the system feeds back these determinations of the patient's head position in the space to the operator and/or patient, aiming to keep the VNG or ENG studies within a defined standard thus guaranteeing the reproducibility of said studies.

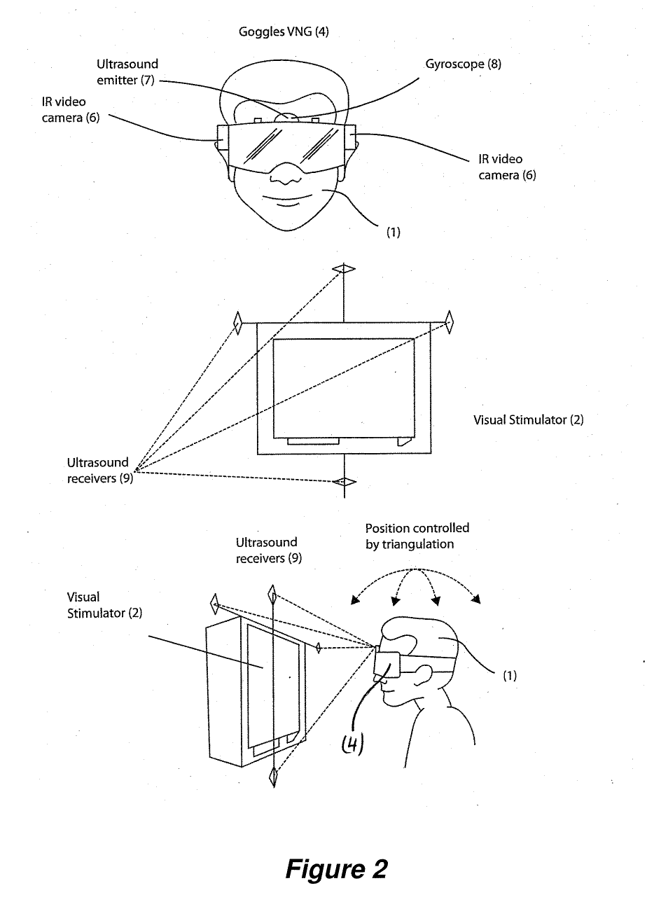

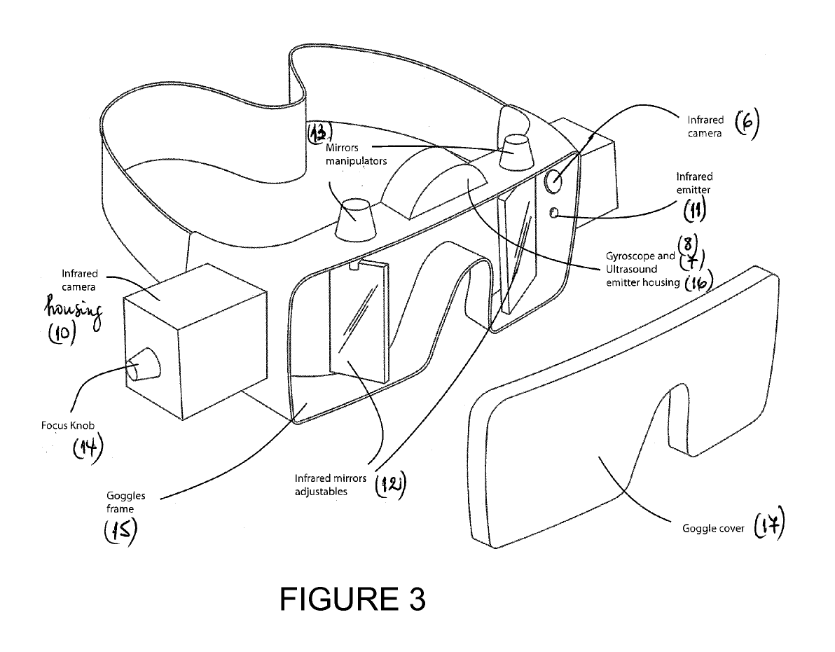

According to FIG. 2, a preferred embodiment of the system of the present invention includes goggles (4) and a visual stimulator (2) connected to a personal computer (PC). FIG. 3, which shows the goggles (4) in more detail, is discussed below.

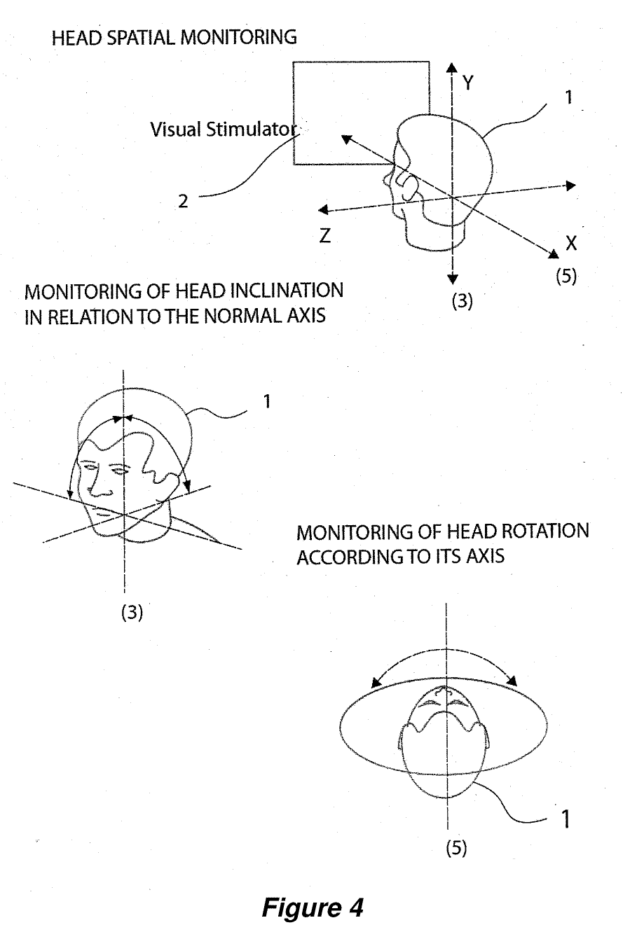

FIG. 4 shows the different types of monitoring of the position and movements of the patient's head (1) that may be carried out by the system according to this invention, said determinations being the ones that allow the standardization of the tests to achieve the reproducibility necessary to be deemed valuable from a diagnostic point of view.

Indeed, the system according to this invention determines and monitors the position and movement of the patient's head (1) in real time. This monitoring can be divided into three different controls:
1) Determination and monitoring of the position of the patient's head (1) in the space in relation to the visual stimulator (2) placed in front of the patient.
2) Determination and monitoring of the inclination of the patient's head (1) in relation to the normal or vertical axis (3) of the patient.
3) Control and monitoring of the rotation of the patient's head (1) in relation to his axis (5).

The information provided by the sensors (7,8) determine and monitor the position, inclination and rotation the patient's head (1) is taken by the system, wherein an algorithm processes the signals and generates instructions for the operator and/or patient as a feedback to keep the tests within the defined standard for each test.

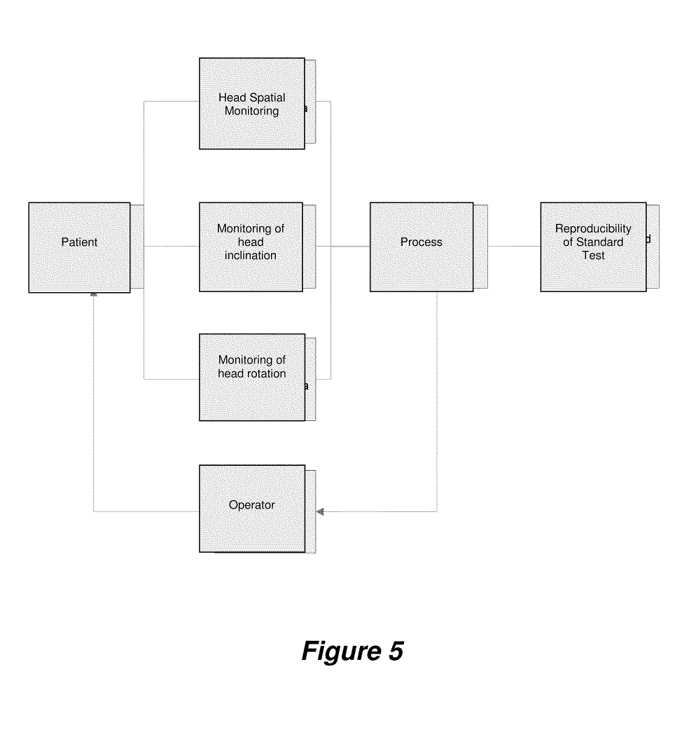

FIG. 5 shows a block diagram that exhibits the interaction between the operator and the patient during the VNG or ENG tests through the system according to this invention.

In the most preferred embodiment of this invention, the portable system for the monitoring and positioning of the patient's head (1) during videonystagmography (VNG) and/or electronystagmography (ENG) studies, which allows the standardization and reproducibility of the said studies, is comprised of: nystagmus detection means (6); means for the determination in real time of the position (7) and movements (8) of the patient's head (1) in space and their transmission through signals; a visual stimulator (2); and a personal computer (PC) (not shown) connected to the nystagmus detection means (6), to the means for the determination in real time of the position (7) and movements (8) of the patient's head (1) and to the visual stimulator (2), which has software to determine the position of the eyes through the analysis of the data generated from the signals emitted by the nystagmus detection means (6); the position and movements of the patient's head in space through the analysis of the data generated from the signals emitted by the means for determining such position (7) and movements (8); wherein said means transmit those data to the PC; wherein the software compares the emitted signal with the signal received for each of the means used and calculates the position of the patient's head (1) in space, wherein the nystagmus detection means (6) and the means for the determination in real time of the position (7) and movements (8) of the patient's head (1) in space are arranged on the patient's head (1) and laterally placed in front of his eyes, while the visual stimulator (2) is arranged before the patient's head (1) and at height of his eyes, further comprising means for the detection (9) of the spatial position of the patient's head (1).

In a preferred embodiment of this invention, the goggles (4) shown in FIG. 3, which can also adopt the form of a mask or a helmet, contain at least two infrared cameras (6) in a housing (10), with illumination of infrared light emitting diodes (11) LED (LED's, acronym of Light-Emitting Diode/s), which fit laterally with regard to the eyes through an adjustable infrared minor (12) allowing vision through, straight to the visual stimulator (2), to permanently monitor the position of the pupil. Seeing that the position of the infrared cameras (6) is laterally placed in relation to the eyes, the centering of the pupils is made through the minor (12). In a preferred embodiment, the cameras (6) are fixed and the mirrors (12) are movable by minor manipulators (13) thus enabling the centering of the pupils.

Each camera (6) comprises an independent focusing control (14) and transmits a signal to the computer which calculates in real time the position of each eye.

Over the goggles frame (15), at least one ultrasound emitter (7) is mounted, which generates an ultrasonic signal that is detected by at least three ultrasound receivers (9), which transmit such signal to the PC, which compares the signal emitted in relation to the one received by each receiver (9) and calculates the position of the patient's head in space. This process is also carried out in real time.

Also mounted on the goggles frame (15), there is at least a gravity vector detector (8), for example a gyroscope, which is used together with the ultrasound signals to determine in real time the angular position and inclination of the patient's head (1).

In one preferred embodiment, both the ultrasound emitter (7) and the gyroscope (8) are mounted in a single housing (16) over the goggles frame.

Ultrasound sensors (9) and emitters (7) are used combined with gravity detectors (8) such as gyroscopes or other necessary sensors and emitters.

Preferably, in one embodiment, at least one gyroscope is used as gravity detector (8).

In another preferred embodiment, only one electronic-type gyroscope (8) is used.

Furthermore, in a preferred embodiment of the invention, the goggles comprise a cover (17) to be placed in front of the patient's eyes to achieve those tests that require non fixation of patient's eyes.

The visual stimulator (2) is placed opposite the patient and it is used for the tests of optical stimuli follow-up by the eyes during the optokinetic tests.

A suitable software commands the PC generating instructions for the operator from the processes of images and positions of the patient.

In this way, the operator may know if the patient is making the maneuvers within the established limits or if he must correct any parameters or positions or give instructions to the patient.

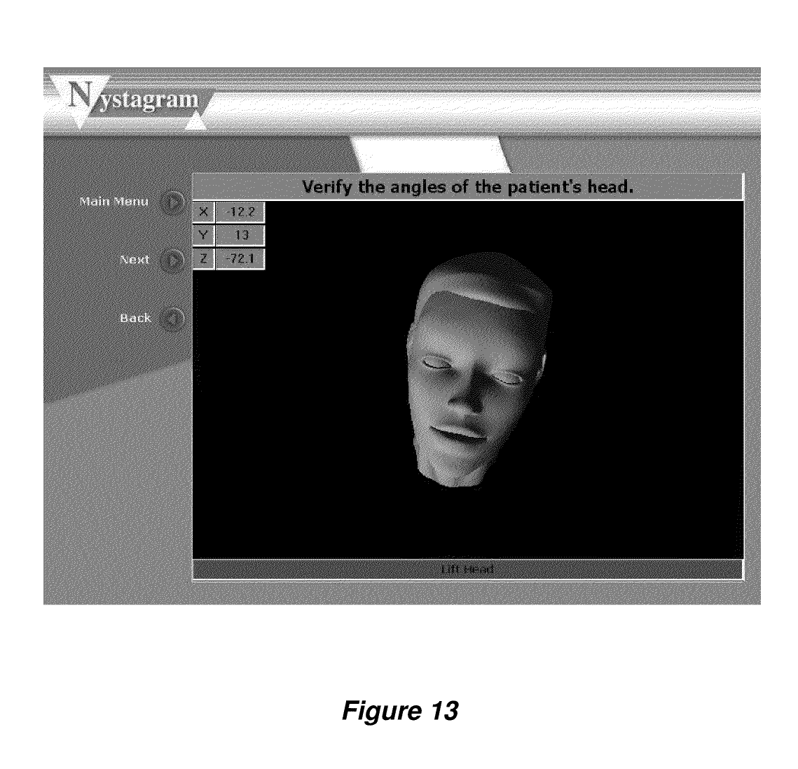

The PC's feedback to the operator consists of visual images of the position of the patient's head (1) and also in an audible way, with alarms and oral instructions. For example, see the screens illustrated in FIGS. 13 to 15.

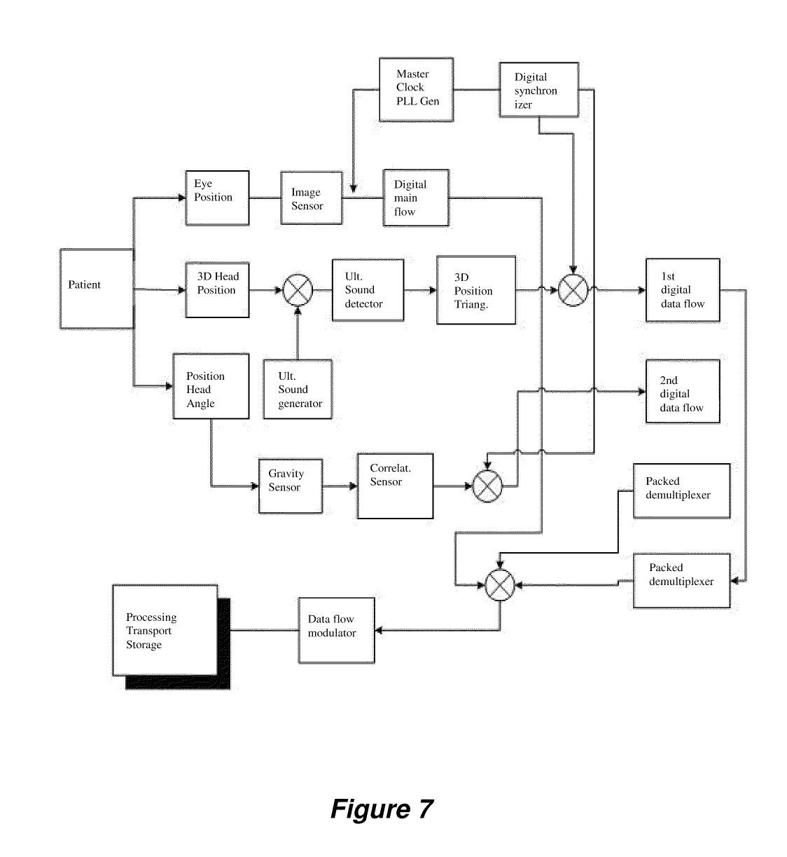

The block diagram in FIG. 7 describes in detail the process of information record, processing and transmission to the PC.

As for the patient, the system determines the position of the eyes by means of cameras (6), the three-dimensional position of the head (1) by means of ultrasound sensors (9) and the angular position of the head (1) by means of the detection of the gravity vector with a gyroscope (8).

All of these signals are combined to generate the corresponding data. Such data are synchronized and modulated to be then transmitted to the computer via a serial connection, for example, in parallel, of the USB type, or equivalent thereof. Preferably, USB-type fast connections are used.

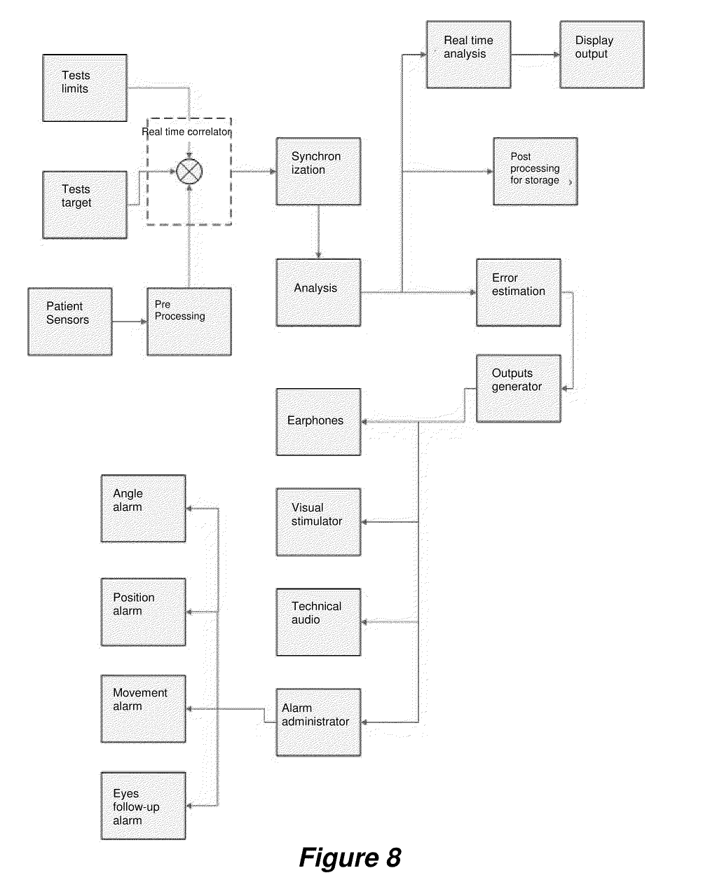

In the diagram block in FIG. 8, the process of signals carried out by the system is described.

The real time correlator device takes the preprocessed signals from the sensors and relates them to the limits imposed in the tests, which are those that determine the desired degree of reproducibility.

Also, the target of the test is correlated, which is an input data that depends on each type of test.

The correlator sends the signal to the synchronization block that adds a base of time to the signals.

With these synchronized information, the analysis is made that has three parallel outputs: the first output is the real time analysis shown on the screen of the operator, the second one is the subsequent process to store the information in the patient's file, and the third one is the error estimation that compares the analyzed signal with the imposed limits.

This error triggers different outputs, sent through the output generator, which are divided into warnings for the operating technician and for the patient, through sounds caught by means of earphones, visual stimuli, audio for the operating technician and alarms.

The alarms are individual and distinctive for each type of violation of the limits previously fixed, such as, for example, angle alarm, position alarm, movement alarm, and alarm of follow-up of eyes.

In the oculomotor test, visual stimuli are generated controlling the patient-stimulator distance, the movements of the patient's head (1), the angles of the patient's head, instructions are provided for the operator about the test technique and its conduction is controlled.

In the postural and positional test or DIX-Hallpike maneuvers, the angles of the patient's head (1) in a dynamic and static way are controlled, instructions are provided for the operator about the test technique and its conduction is controlled.

In the caloric stimulation test, the static angles of the patient's head (1) and the movements of the patient's head (1) are controlled, instructions are provided for the operator about the test technique and its conduction is controlled.

In the rotatory test (AHR), the patient-stimulator distance, the movements of the patient's head (1) and the angles of the patient's head (1) are controlled, instructions are provided for the operator about the test technique and its conduction is controlled.

During the conduction of the tests, the system according to the present invention determines the angles and distances by means of ultrasound triangulation devices and a gyroscope as gravitational sensor. The gyroscope (8) is capable of measuring the components of the gravity vector with respect to the reference of the sensor and dynamic variations of acceleration.

The ultrasound detectors (9) together with the gyroscope (8) send their signals to a PC which processes the information by means of an algorithm that calculates the three-dimensional position and the speed of movement in real time of the patient's head (1).

The software includes two parts, the positioning algorithm which calculates the position and the speed, and the program that controls the VNG tests.

The VNG system software of the present invention uses the information of the patient's positioning algorithm to generate alarms warning when the position and/or speed of the patient's head (1) is out of the accepted range of the test that is being conducted.

The equipment rejects the results of badly made determinations and only accepts the tests that are made within the admitted ranges, thus generating a safety net which ensures that the operator and the patient have made the maneuvers of stimulation and taking of data in an adequate, reproducible and consistent way.

The feedback provided in real time in the form of alarms and visual guides helps and guides the operator in the conduction of the tests.

The software is expected to have the capacity to generate images that guide the operator at any moment and whichever test is made.

FIG. 13 shows a screen generated by the software that verifies the static angular position of the patient's head (1) in real time.

In this way, the position of the patient's head (1) can be verified and recommendations are provided for the operator.

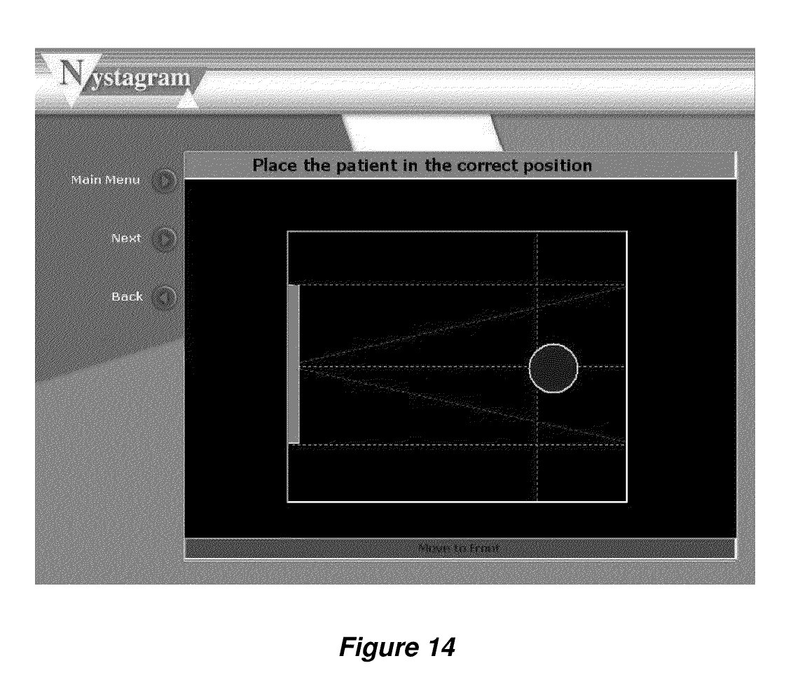

FIG. 14 shows a screen generated by the software that verifies the angular position of the patient's head (1) in real time in front of the visual stimulator (2).

In the case that the conduction of a test needs verification with correction suggestions of the patient's head (1) positioning, the software can provide the corrections from the entering data.

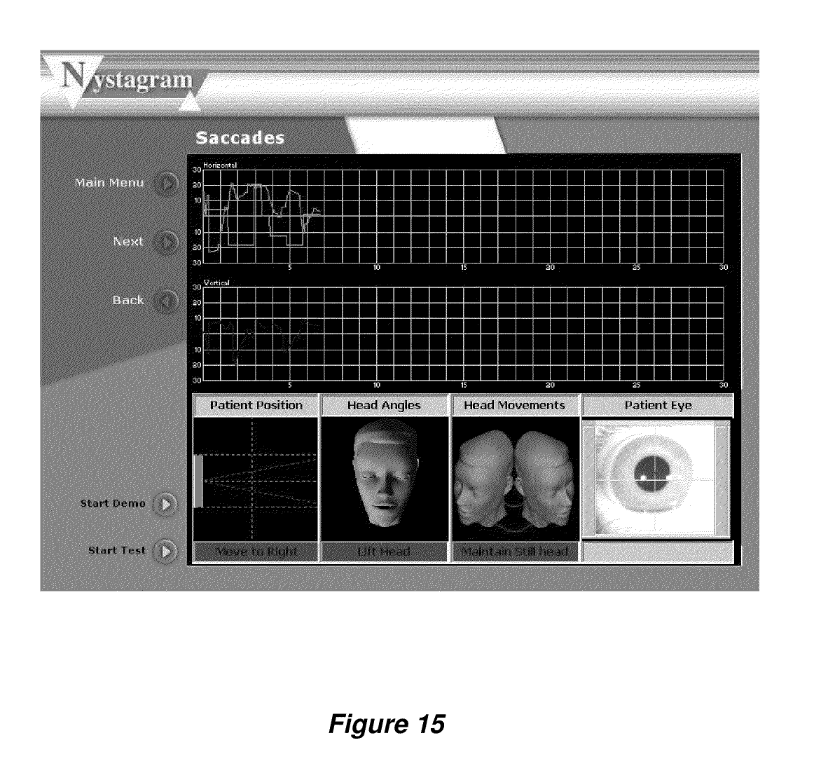

For example, the position control regarding the visual stimulator (2), the angular position of the patient's head (1) and the angular motion of the patient's head (1), with alarm, for example in red, of movements or positioning outside the reproducibility range, along with the monitoring of the eye movement in real time, and a graphical representation of the eye movement in horizontal and vertical channels, can be obtained in a single screen as shown in FIG. 15.

From the foregoing, it could be seen that the system of the present invention provides the operator and/or the patient with instructions on how to proceed during the tests according to the data provided by the sensors and processed by the computer on a constant feed-back basis.

Therefore, the description is a portable system that may be used in any physician's office. The system is easy to move which characterizes its portability aspect since it may be placed within a briefcase, may be easily carried and it is externally connected to a portable computer such as a “laptop” or “notebook” or to a desktop computer existing in the place where it is to be used.

This system has the advantage that it may be used by a person with a basic training or little knowledge of the used techniques owing to the fact that the system provides parameters, alarms, warnings and records of everything that has been carried out.

Likewise, this system may be used as an “up-grade” of other preexisting equipment. That is to say that it may be associated with existing videonystagmographs (VNG) and/or electronystagmographs (ENG) in order to provide them with the advantage of being operated by a non-professional technician since the system provides instructions, test parameters and data obtained in a standardized way.

The above-given description may be evidently modified by any person skilled in the art, without being construed as a departure from the scope of the claims accompanying this patent.

The claims are part of the description of this invention.

What is claimed is:

1. A portable system for monitoring of a patient's head positioning during videonystagmography (VNG) or electronystagmography (ENG) studies, comprising:
a visual stimulator; and
a personal computer (PC), wherein
the PC determines position of the patient's head in space, the position comprising position in relation to the visual stimulator placed before the patient's head, inclination of the patient's head in relation to the patient's vertical axis (normal) and rotation of the patient's head in relation to the patient's vertical axis (normal), and the PC re-feeds the determinations of the position of the patient's head in space to an operator or the patient keeping the VNG or ENG studies within a defined standard.

2. A portable system for monitoring of a patient's head positioning during videonystagmography (VNG) or electronystagmography (ENG) studies standardizing and enabling reproduction of the studies, the system comprising:
nystagmus detection means emitting a signal;
means for determining in real time position and movements of the patient's head in a space and transmitting the position and movements of the patient's head in the space through signals;
means for detection of a special position of the patient's head;
a visual stimulator; and
a personal computer (PC) connected to the nystagmus detections means, to the means for determining in real time the position and movements of the patient's head in the space and to the visual stimulator, the PC receiving the signals transmitted by the means for determining in real time position and movements of the patient's head in the space, the PC having a software that determines a position of the patient's eyes through analysis of data generated from the signals emitted by the nystagmus detection means, and determines the position and movements of the patient's head in the space through analysis of the data generated from the signals transmitted by the means for determining in real time position and movements of the patient's head in space, and the software compares the emitted signal to the signal received from the means for determining in real time position and movements of the patient's head in the space for each of the means used and calculates the position of the patient's head in the space,
wherein the nystagmus detection means and the means for determining in real time the position and movements of the patient's head in the space are arranged on the patient's head and near the patient's eyes, and the visual stimulator is arranged in front of the patient's head and at a certain height and distance from the patient's eyes.

3. The system of claim 2, wherein the nystagmus detection means are infrared cameras arranged to detect the position of a patient's pupil or electrodes arranged around the patient's eyes.

4. The system of claim 3, wherein the means for determining in real time position and movements of the patient's head in the space comprises at least two infrared cameras, with infrared Light Emitting Diodes (LED), and the infrared cameras are adjusted laterally with respect to the patient's eyes.

5. The system of claim 2, wherein the means for determining in real time the position and movements of the patient's head in the space are comprised of at least one ultrasound emitter that transmits ultrasonic signals.

6. The system of claim 2, wherein the means for determining in real time the angular position and inclination of the patient's head are comprised of at least a gyroscope as detector of gravity vector mounted on the patient's head, the gyroscope transmits signals that are used together with the ultrasound signals.

7. The system of claim 2, 3, 4, 5 or 6, wherein the nystagmus detection means and the means for determining in real time the position and movements of the patient's head in the space are arranged on the patient's head mounted on a pair of goggles, headband, mask or helmet.

8. The system of claim 2, wherein the visual stimulator is a set of lights arranged in a plane, a display of cathode ray tube, a liquid crystal display, or equivalent thereof.

9. The system of claim 8, wherein the visual stimulator is comprised of a display that shows images corresponding to a test and has at least three ultrasound detectors, one arranged above the stimulator and the other two at both sides of the stimulator.

10. The system of claim 2, wherein the data are synchronized and modulated for subsequent transmission to the PC through a serial connection or equivalent thereof.

11. The system of claim 10, wherein the serial connection is one or more of in parallel, a USB type of connection, or equivalent thereof.

12. The system of claim 2, wherein the visual stimulator is comprised of four ultrasound receivers, where one ultrasound receiver is mounted on an upper part, two ultrasound receivers are mounted laterally and a last ultrasound receiver is mounted on a lower part of the visual stimulator.

13. The system of claim 2, wherein the visual stimulator monitors tests of optical stimuli of the patient's eyes during Optokinetic tests.

14. The system of claim 2, wherein the software generates instructions for an operator in a display, taking into account data analysis of image processes and positions adopted by the patient.

15. The system of claim 2, wherein the software provides an operator with visual images of the position of the patient's head and audible feedback.

16. The system of claim 15, wherein the software provides the operator with an audible feedback through alarms and oral instructions.

17. The system of claim 1 or 2, enabling performance of VNG or ENG studies allowing exploration of vestibular system of the patient.

18. The system of claim 17, wherein VNG studies consist of a selected subgroup of tests comprising at least Oculomotor or Optokinetic tests (Saccades, Smooth Pursuit, OKN, Gaze and Spontaneous Nystagmus), Postural and Positional tests (Positional and Positioning; Dix-Hallpike Maneuvers), Caloric Stimulation tests, Head Active Movements tests (Head Trust), Rotatory tests (Active Head Rotatory (AHR)/Rotatory), and Spontaneous tests.

19. A portable system for monitoring of a patient's head positioning during videonystagmography (VNG) or electronystagmography (ENG) standardizing and enabling reproduction of the studies, the system comprising:
a pair of goggles comprised of at least two infrared cameras, with infrared light emitting diodes to determine in real time position of a patient's eyes through signal transmission, where the infrared cameras are adjusted laterally with respect to the patient's eyes while pupils of the patient's eyes are being centered by means of mirrors and focused by individual focus control of each camera; at least one ultrasound emitter mounted on the goggles to determine in real time the position of the patient's head in space through ultrasonic signal transmission; and at least a gyroscope as detector of gravity vector mounted on the goggles to determine in real time angular position and inclination of the patient's head through the signal transmission together with the ultrasound signals;
a visual stimulator comprised of a display that shows images corresponding to a test and at least three ultrasound detectors, one of the ultrasound detectors is arranged in an upper part of the stimulator and the other two are arranged at both sides of the stimulator; and
a personal computer (PC) connected to the goggles and the visual stimulator, the PC is comprised of a software that determines the position of the patient's eyes through analysis of data generated from signals emitted by the infrared cameras; an angular position of the patient's head through analysis of data generated from signals emitted by the detector of gravity vector; and a three-dimensional position of the patient's head through analysis of data generated from signals emitted by the ultrasound emitter and detected by ultrasound receivers, which, in turn, transmit the signal to the PC, where the emitted signal is compared to the received signal of each ultrasound receiver, and where the position of the patient's head in the space is also calculated;
wherein, the goggles are arranged on the patient's head and in front of the patient's eyes, while the visual stimulator is arranged before the patient's head at a height of the patient's eyes.
